(19) United States
(12) Patent Application Publication
(10) Pub. No.: US 2026/0165664 A1
(43) Pub. Date: Jun. 18, 2026

(54) X-RAY CT APPARATUS AND IMAGE PROCESSING METHOD

(71) Applicant: CANON MEDICAL SYSTEMS CORPORATION, Otawara-shi (JP)

(72) Inventor: Ryusei SAIKI, Nasushiobara (JP)

(73) Assignee: CANON MEDICAL SYSTEMS CORPORATION, Otawara-shi (JP)

(21) Appl. No.: 19/415,911

(22) Filed: Dec. 11, 2025

(30) Foreign Application Priority Data
Dec. 13, 2024 (JP) 2024-218679

(51) Int. Cl. A61B 6/03; A61B 6/00; A61B 6/02; A61B 6/04; A61B 6/46

(52) U.S. Cl. CPC A61B 6/035; A61B 6/027; A61B 6/0407; A61B 6/465; A61B 6/5205; A61B 6/547

(57) ABSTRACT

An X-ray computed tomography (CT) apparatus according to the present embodiment includes an identification unit, a determination unit, and a generation unit. The identification unit identifies gantry state information indicating a state of a gantry, the gantry state information including at least one of orientation information indicating an orientation of the gantry having an opening, and object information indicating an object other than a subject, the object existing in the opening. The determination unit determines an image quality adjustment parameter to be used in reconstruction processing, based on the identified gantry state information, and parameter information associating the gantry state information with the image quality adjustment parameter including a plurality of parameters for determining image quality of a reconstructed image generated through reconstruction processing. The generation unit generates the reconstructed image by executing the reconstruction processing on first detection data collected by emitting an X-ray toward the subject using the determined image quality adjustment parameter.

### CROSS-REFERENCE TO RELATED APPLICATIONS

This application is based upon and claims the benefit of priority from Japanese Patent Application No. 2024-218679, filed Dec. 13, 2024, the entire contents of which are incorporated herein by reference.

### FIELD

Embodiments described in this specification and drawings relate to an X-ray computed tomography (CT) apparatus and an image processing method.

### BACKGROUND

There has been conventionally known an X-ray computed tomographic imaging apparatus (hereinafter also referred to as an X-ray computed tomography (CT) apparatus) capable of capturing an image of a subject in a supine position or a standing position.

On the other hand, in the X-ray CT apparatus, various parameters for adjusting image quality of a CT image (hereinafter also referred to as image quality adjustment parameters) are used to obtain a CT image with image quality desired by the user. As the image quality adjustment parameters, correction data for adjusting the image quality based on pixel values for water and air, an X-ray tube alignment indicating an arrangement of an X-ray tube provided on a gantry, an image quality parameter that determines image quality of a CT image, and the like are known.

In the conventional X-ray CT apparatus capable of capturing an image of a subject in the supine position or the standing position, to adjust the image quality of a CT image, image quality adjustment parameters suitable for an image capturing condition have been used irrespective of an image capturing body position (image capturing mode) of the subject. However, because the influence of gravity on a unit including an X-ray tube, an X-ray detector, and the like inside the gantry is different between a case where an image is captured in a supine position mode and a case where an image is captured in a standing position mode, it has been known that a difference arises in collected X-ray data.

Accordingly, even in a case where CT images are captured under the same image capturing condition, a difference might arise in the image quality between the obtained CT images depending on whether the image capturing mode is the supine position mode or the standing position mode.

### BRIEF DESCRIPTION OF THE DRAWINGS

FIG. 1 is a diagram illustrating an example of a configuration of an X-ray computed tomography (CT) apparatus according to an embodiment;

FIG. 2 is a perspective view illustrating an example of a state of a gantry device in a standing position mode according to the embodiment;

FIG. 3 is a perspective view illustrating an example of a state of the gantry device in a supine position mode according to the embodiment;

FIG. 4 is a diagram illustrating an example of calculation processing of an image quality adjustment parameter according to the embodiment;

FIG. 5 is a diagram illustrating an example of calculation processing of an image quality adjustment parameter according to the embodiment;

FIG. 6 is a diagram illustrating an example of determination of an image quality adjustment parameter and reconstruction processing that uses the image quality adjustment parameter according to the embodiment;

FIG. 7 is a diagram illustrating an example of determination of an image quality adjustment parameter and reconstruction processing that uses the image quality adjustment parameter according to the embodiment;

FIG. 8 is a flowchart illustrating an example of calculation and storing processing of an image quality adjustment parameter according to the embodiment; and

FIG. 9 is a flowchart illustrating an example of determination and reconstruction processing of the image quality adjustment parameter that uses the image quality adjustment parameter according to the embodiment.

### DETAILED DESCRIPTION

An X-ray computed tomography (CT) apparatus according to the present embodiment includes an identification unit, a determination unit, and a generation unit. The identification unit identifies gantry state information indicating a state of a gantry, the gantry state information including at least one of orientation information indicating an orientation of the gantry having an opening, and object information indicating an object other than a subject, the object existing in the opening. The determination unit determines an image quality adjustment parameter to be used in reconstruction processing, based on the identified gantry state information, and parameter information associating the gantry state information with the image quality adjustment parameter including a plurality of parameters for determining image quality of a reconstructed image generated through reconstruction processing. The generation unit generates the reconstructed image by executing the reconstruction processing on first detection data collected by emitting an X-ray toward the subject using the determined image quality adjustment parameter.

Various Embodiments will be described hereinafter with reference to the accompanying drawings.

The X-ray CT apparatus according to the present embodiment has a structure in which the orientation of the gantry can be changed between a standing position image capturing state in which an image of a subject P in a standing position can be captured and a supine position image capturing state in which an image of the subject P in a supine position can be captured. In the following embodiment, components denoted by the same reference numerals are assumed to perform similar operations, and redundant descriptions are omitted as appropriate.

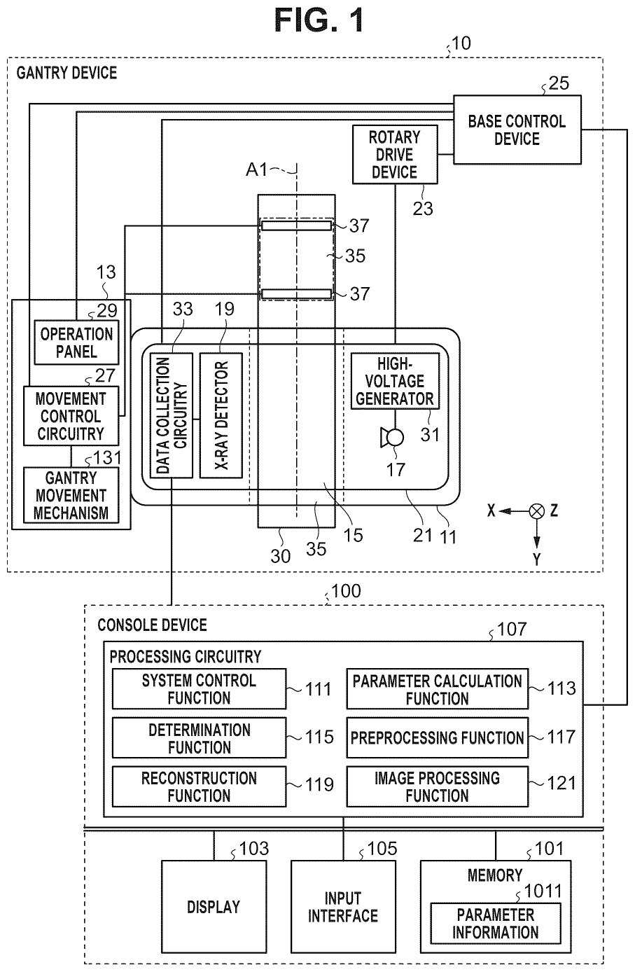

FIG. 1 is a diagram illustrating a configuration example of an X-ray CT apparatus 1 according to an embodiment. As illustrated in FIG. 1, the X-ray CT apparatus 1 includes a gantry device 10 and a console device 100. For example, the gantry device 10 is installed in a CT examination room, and the console device 100 is installed in a control room adjacent to the CT examination room. The gantry device 10 and the console device 100 are connected in a wired or wireless manner so as to be able to communicate with each other.

In the present embodiment, an axis direction perpendicular to a floor surface, i.e., a vertical direction, is defined as a Z-axis direction, and two directions orthogonal to the Z-axis direction and orthogonal to each other are defined as an X-axis direction and a Y-axis direction.

The gantry device 10 is a scanning apparatus having a configuration for performing X-ray CT image capturing of the subject P in a standing position or a supine position. The console device 100 is a computer that controls the gantry device 10.

The gantry device 10 includes a gantry 11, a support column 13, a rotary drive device 23, and a gantry control device 25.

The gantry 11 includes an image capturing system related to image capturing of the subject P, and an opening 15 into which the subject P can be inserted. The support column 13 supports the gantry 11 in such a manner that the direction of the opening 15 can be changed between a vertical direction and a horizontal direction and that the gantry 11 can be moved in the vertical direction.

In FIG. 1, the gantry 11 is supported by the support column 13 as a cantilever; however, this is not limiting. For example, the gantry 11 may be supported by a plurality of support columns (e.g., two support columns). The support column 13 may be referred to as a support column portion.

The gantry 11 has the opening 15 constituting an image capturing space related to the image capturing of the subject P. The gantry 11 is a substantially cylindrical structure in which the opening 15 is formed. As illustrated in FIG. 1, the gantry 11 houses an X-ray tube 17 and an X-ray detector 19 arranged so as to face each other across the opening 15. The X-ray tube 17 and the X-ray detector 19 are included in the image capturing system related to the image capturing of the subject P in the present embodiment.

The image capturing system may further include data collection circuitry (hereinafter also referred to as a data acquisition system (DAS)) 33, a high-voltage generator 31, a collimator, a wedge, and the like. In other words, the gantry 11 includes the image capturing system related to the image capturing of the subject P. The gantry 11 is supported by the support column 13 so as to be movable in the vertical direction along the support column 13.

In addition, the gantry 11 is supported by the support column 13 such that the direction of the opening 15 can be changed between the vertical direction and the horizontal direction. The direction of the opening 15 corresponds to, for example, a direction in which a couchtop 30 is inserted into the opening 15, i.e., a direction extending along a rotational axis A1.

The gantry 11 includes a main frame (not illustrated) formed of metal such as aluminum, and a rotational frame 21 supported by the main frame so as to be rotatable about the rotational axis A1 via a bearing or the like. At a contact portion between the main frame and the rotational frame 21, an annular electrode (not illustrated) is provided. A conductive slider (not illustrated) is attached to the contact portion of the main frame so as to come into sliding contact with the annular electrode.

The support column 13 is a base member that supports the gantry 11 at a distance from the floor surface.

The support column 13 has a columnar shape such as a cylindrical shape or a prismatic shape, for example. The support column 13 is formed of a material such as plastic or metal, for example. The support column 13 is attached to a lateral surface portion of the gantry 11, for example. The support column 13 supports the gantry 11 in a state in which the rotational axis A1 of the opening 15 is oriented substantially perpendicular to the floor surface in such a manner that the gantry 11 is slidable in the vertical direction, to perform X-ray CT image capturing of the subject P in a seated position or a standing position.

Typically, the support column 13 is provided on one lateral side of the gantry 11. However, the present embodiment is not limited thereto. For example, two support columns 13 may be connected to both lateral sides of the gantry 11. In other words, at least one of the support columns 13 supports the gantry 11 to be movable in the vertical direction.

While the support column 13 is described to have a columnar shape, the present embodiment is not limited thereto. For example, the support column 13 may have any shape such as a U-shape as long as at least one of the lateral sides of the gantry 11 can be supported.

The support column 13 supports the gantry 11 such that the rotational axis A1 is rotatable about a horizontal axis (hereinafter also referred to as a tilt axis) parallel to the floor surface, between the vertical direction and the horizontal direction. The support column 13 and the gantry 11 are connected such that the gantry 11 is rotatable about the tilt axis, for example, via a slewing bearing.

Specifically, a linear guide is provided on the support column 13 along the vertical direction. A slewing bearing is provided on a block movable along the linear guide. The block moves along the linear guide by driving of a motor under the control of movement control circuitry 27.

A gear that meshes with a gear (internal gear) of the slewing bearing is connected to a rotational axis of a motor via various gears or the like that generate a predetermined torque. The internal gear of the slewing bearing rotates by driving of the motor under the control of the movement control circuitry 27.

Accordingly, the gantry 11 is rotatable about an X-axis that serves as a rotational axis in FIG. 1 and is movable in the vertical direction. The above-described linear guide and the slewing bearing correspond to a gantry movement mechanism 131 related to movement of the gantry 11. In other words, the gantry movement mechanism 131 is mounted on the support column 13.

The gantry movement mechanism 131 moves the gantry 11 by moving the block along the linear guide arranged along the vertical direction, under the control of the movement control circuitry 27. The gantry 11 accordingly becomes movable up and down along the vertical direction. A mechanism related to the movement of the gantry 11 along the vertical direction is not limited to the linear guide or the like, and may be implemented, for example, by a known mechanism such as a rack-and-pinion.

In addition, under the control of the movement control circuitry 27, the gantry movement mechanism 131 rotates the gantry 11 between the horizontal direction and the vertical direction by the rotation of the internal gear of the slewing bearing.

A rotation mechanism that rotates the gantry 11 is not limited to the slewing bearing, and may be implemented by a known mechanism. The rotation of the gantry 11 by the rotation mechanism enables switching between a standing position or seated position image capturing state (also referred to as a standing position mode) and a supine position image capturing state (also referred to as a supine position mode), i.e., switching between the standing position mode and the supine position mode.

For example, in a case where supine position image capturing of the subject P is performed, the gantry movement mechanism 131 rotates the gantry 11 such that the opening 15 becomes vertical under the control of the movement control circuitry 27. After the subject P lies down on the couchtop 30, the couchtop 30 is horizontally moved by a couchtop movement mechanism 37 to be described below, and thereby it becomes possible to perform the supine position image capturing of the subject P in a manner similarly to that of a normal X-ray CT apparatus.

In the case where standing position image capturing of the subject P is performed, the rotation mechanism of the gantry movement mechanism 131 rotates the gantry 11 such that the opening 15 becomes horizontal under the control of the movement control circuitry 27. The subject P stands in a state where his or her back resting against the support column 13, and the standing position image capturing is performed by vertically moving the gantry 11. In the standing position mode, the couchtop movement mechanism 37 to be described below retracts the couchtop 30 to a position where the couchtop 30 does not interfere with the gantry 11, under the control of the movement control circuitry 27.

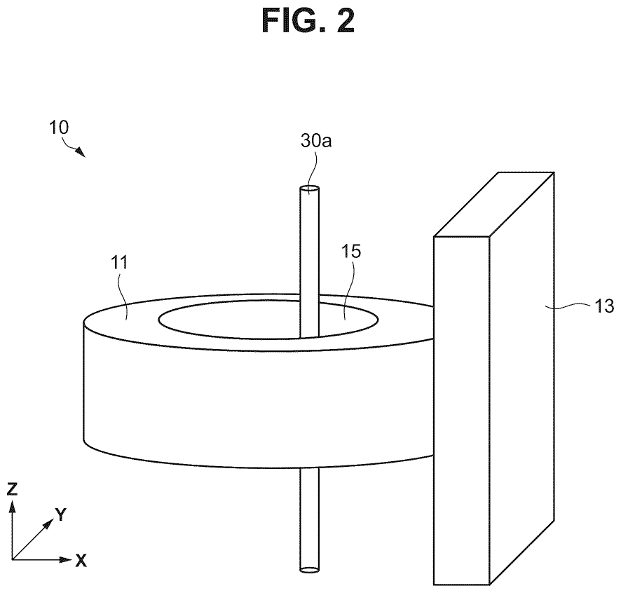

FIG. 2 is a perspective view illustrating an example of a state of the gantry device 10 in the standing position mode. As illustrated in FIG. 2, in the present embodiment, in the standing position mode, the standing position image capturing of the subject P is performed in a state where a columnar subject holding tool (also referred to as a patient fixation pole) 30a exists inside the gantry 11. By standing with his or her back lightly resting against the patient fixation pole 30a, the subject P can maintain a stable standing position without swaying.

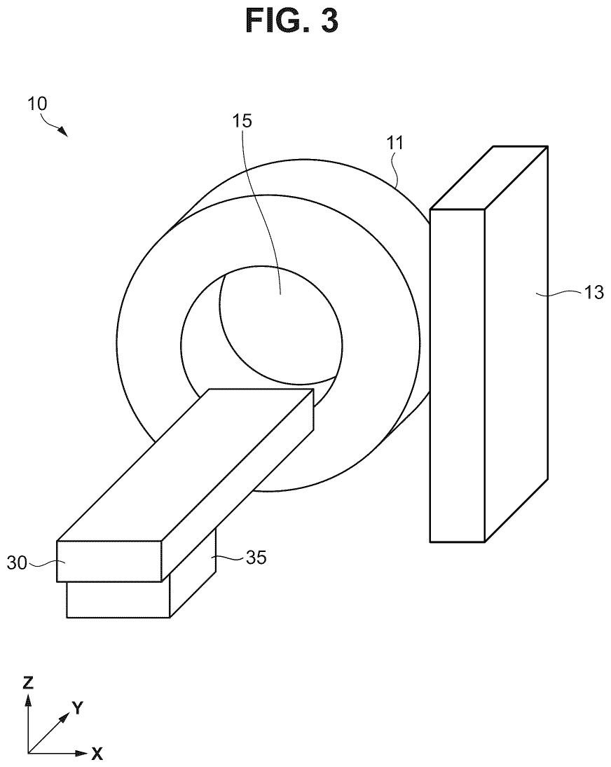

FIG. 3 is a perspective view illustrating an example of a state of the gantry device 10 in the supine position mode. As illustrated in FIG. 3, in the supine position mode, the couchtop 30 is supported in a horizontal state on a base 35 via the couchtop movement mechanism 37. At this time, the couchtop 30 is freely movable along a longitudinal direction of the couchtop 30 under the control of the movement control circuitry 27.

The X-ray tube 17 is a vacuum tube that generates X-rays by emitting thermal electrons from a cathode (filament) toward an anode (target) through the application of high voltage from the high-voltage generator 31 and the supply of filament current. By the thermal electrons colliding with the target, X-rays are generated. X-rays generated at a focal spot of the X-ray tube 17 are shaped into a cone beam shape via a collimator, for example, and emitted toward the subject P.

For example, the X-ray tube 17 may be a rotating anode type X-ray tube that generates X-rays by emitting thermal electrons toward a rotating anode. In the present embodiment, the configuration is applicable not only to a single-tube type X-ray CT apparatus, but also to what is called a multi-tube type X-ray CT apparatus in which a plurality of pairs of X-ray tubes 17 and X-ray detectors 19 are mounted on the rotational frame 21.

The X-ray detector 19 detects X-rays that have been emitted from the X-ray tube 17 and transmitted through the subject P, and outputs an electric signal corresponding to an X-ray intensity to the DAS 33. The X-ray detector 19 includes a plurality of detection element columns in which a plurality of detection elements is arrayed in a channel direction along one arc centered on a focus of the X-ray tube 17, for example. The X-ray detector 19 has a structure in which the plurality of detection element columns is arrayed in a slice direction (column direction, row direction), for example.

Types of the X-ray CT apparatus 1 include a rotate/rotate-type (third-generation CT) in which the X-ray tube 17 and the X-ray detector 19 integrally rotate around the subject P, and a stationary/rotate-type (fourth-generation CT) in which a large number of X-ray detection elements arrayed in a ring shape are fixed and only the X-ray tube 17 rotates around the subject P. Both of the types are applicable to the present embodiment. Hereinafter, to provide a specific description, the X-ray CT apparatus 1 according to the present embodiment will be described by taking the third-generation CT as an example.

The X-ray detector 19 is, for example, an indirect conversion type detector including a grid, a scintillator array, and an optical sensor array. The scintillator array includes a plurality of scintillators, and each scintillator includes a scintillator crystal that outputs light with a photon quantity corresponding to an incident X-ray intensity. The grid is arranged on an X-ray incident surface of the scintillator array, and includes an X-ray shielding plate having a function of absorbing scattered X-rays.

The grid may also be referred to as a collimator (one-dimensional collimator or two-dimensional collimator). The optical sensor array has a function of converting the amounts of light from scintillators into electric signals, and includes an optical sensor such as a photomultiplier (PMT), for example.

The X-ray detector 19 may be a direct conversion type detector including a semiconductor device that converts incident X-rays into electric signals. The X-ray detector 19 may be a photon counting type X-ray detector.

The rotational frame 21 has the opening 15, and the X-ray tube 17 that generates X-rays is attached thereto. Specifically, the rotational frame 21 is an annular frame that supports the X-ray tube 17 and the X-ray detector 19 to face each other and rotates the X-ray tube 17 and the X-ray detector 19 by the gantry control device 25 to be described below.

The rotational frame 21 is rotatably supported on the main frame via a support bearing. The rotational frame 21 rotates about the rotational axis A1 at a fixed angular velocity by receiving driving power from the rotary drive device 23 under the control of the gantry control device 25.

The rotational frame 21 further includes and supports the high-voltage generator 31 and the DAS 33 in addition to the X-ray tube 17 and the X-ray detector 19. Such a rotational frame 21 is housed in a substantially cylindrical casing in which the opening 15 constituting an image capturing space is formed. The central axis of the opening 15 coincides with the rotational axis A1 of the rotational frame 21.

Detection data generated by the DAS 33 is transmitted, for example, through optical communication from a transmitter including a light-emitting diode (LED) to a receiver including a photodiode that is provided in a non-rotational portion (e.g., main frame) of the gantry device 10, and is transferred to the console device 100.

A transmission method of detection data from the rotational frame 21 to the non-rotational portion of the gantry device 10 is not limited to the above-described optical communication, and any method may be employed as long as the transmission is non-contact data transmission.

The rotary drive device 23 generates driving power for rotating the rotational frame 21 under the control of the gantry control device 25. The rotary drive device 23 generates the driving power by being driven at a rotational speed corresponding to a duty ratio or the like of a drive signal from the gantry control device 25. The rotary drive device 23 is implemented, for example, by a motor such as a direct-drive motor or a servo motor. The rotary drive device 23 is housed, for example, in the gantry 11.

The gantry control device 25 controls the high-voltage generator 31, the rotary drive device 23, the movement control circuitry 27, and the DAS 33 in accordance with a command from the console device 100. The gantry control device 25 has a function of performing operation control of the gantry device 10 upon receiving an input signal from an input interface attached to the console device 100 or the gantry device 10.

For example, the gantry control device 25 performs the control of rotating the rotational frame 21 upon receiving an input signal, the control of tilting the gantry device 10, and the like. The gantry control device 25 may be provided on the support column 13 in the gantry device 10, or may be provided in the console device 100. In addition, a function implemented by the gantry control device 25 may be incorporated as a gantry control function in processing circuitry 107 of the console device 100.

The gantry control device 25 includes, as hardware resources, a processing device (processor) such as a central processing unit (CPU) or a micro processing unit (MPU), and a storage device (memory) such as a read only memory (ROM) or a random access memory (RAM).

In addition, the gantry control device 25 may be implemented by an application specific integrated circuit (ASIC), a field programmable gate array (FPGA), another complex programmable logic device (CPLD), or a simple programmable logic device (SPLD).

The processing device implements the above-described function by reading out a program stored in the storage device and executing the program. Instead of storing a program in the storage device, a program may be directly installed in circuitry of the processing device. In this case, the processing device implements the above-described function by reading out a program installed in the circuitry and executing the program.

The couchtop 30, on which the subject P can be placed in the supine position mode, can be inserted into the opening 15. The couchtop 30 is supported on the base 35 via the couchtop movement mechanism 37. Specifically, the couchtop 30 is supported by couchtop movement mechanisms 37 provided at both ends in the Y-axis direction of the base 35.

By the couchtop movement mechanisms 37, the couchtop 30 becomes movable in a direction in which the opening 15 penetrates. In other words, the couchtop 30 is fixed in a slidably movable manner relatively to the gantry 11 along the rotational axis A1 of the rotational frame 21 in the image capturing system, via the couchtop movement mechanisms 37.

The couchtop movement mechanisms 37 move the couchtop 30 under the control of the movement control circuitry 27.

Each couchtop movement mechanism 37 includes, for example, a roller guide or the like. The couchtop movement mechanism 37 can be implemented by a known configuration such as a friction drive or a belt mechanism. The couchtop movement mechanism 37 is not limited to a roller guide, a friction drive, a belt mechanism, or the like, and can be appropriately implemented by a known mechanism.

The couchtop movement mechanism 37 may be mounted on an up-down movement mechanism. The up-down movement mechanism includes, for example, the couchtop movement mechanism 37 and is provided on the base 35. The up-down movement mechanism allows the couchtop 30 to move in a direction perpendicular to a placement surface of the subject P on the couchtop 30.

For example, the up-down movement mechanism is implemented by an actuator (e.g., of a piston type) that can move (push up) a rotational axis of the roller guide in a Y-axis direction. An implementation method of the up-down movement mechanism is not limited to the actuator.

In addition, a left-right movement mechanism may be provided between the couchtop movement mechanism 37 and the couchtop 30. For example, couchtop support members covering a bottom surface of the couchtop 30 and lateral surfaces of the couchtop 30 are provided on the bottom surface and the lateral surfaces. The left-right movement mechanism includes a block, a ball screw, a motor, and a belt. The ball screw extends along a short axis direction of the couchtop 30. The block is attached to the ball screw.

The couchtop support member is coupled to the block. Rotational force generated by the motor is transmitted to the ball screw via the belt. When the motor rotates under the control of the movement control circuitry 27, the rotational force generated by the motor is transmitted to the ball screw. The ball screw thereby rotates. In accordance with the rotation of the ball screw, the block moves in the short axis direction of the couchtop 30.

The movement control circuitry 27 controls movement of the gantry 11 and the couchtop 30.

For example, in a case where the user issues an instruction to capture an image of the subject P in the standing position mode, the movement control circuitry 27 controls the gantry movement mechanism 131 to rotate the gantry 11 such that the opening 15 becomes vertical.

In addition, for example, in the case where the user issues an instruction to capture an image of the subject P in the standing position mode, the movement control circuitry 27 controls the couchtop movement mechanism 37 so as to retract the couchtop 30 to a position where the couchtop 30 does not interfere with the gantry 11 even when the gantry 11 moves in the vertical direction.

A movement mechanism such as a caster may be provided on the base 35. In this case, the couchtop 30 may be retracted by the user manually moving the base 35, or the couchtop 30 may be retracted by automatically moving the base 35 by a drive source such as a motor.

The movement control circuitry 27 is implemented by the above-described processor or the like.

While, in FIG. 1, the movement control circuitry 27 is mounted on the support column 13, the movement control circuitry 27 may be mounted on the gantry 11, or may be mounted on the console device 100. In addition, a function implemented by the movement control circuitry 27 may be mounted on the processing circuitry 107 or the gantry control device 25 as a movement control function.

An operation panel 29 is implemented by a switch button, a touch pad for performing an input operation by touching an operation surface thereof, a touch panel display in which a display screen and a touch pad are integrated, or the like. The operation panel 29 converts an input operation received from the user into an electric signal, and outputs the electric signal to the gantry control device 25.

The operation panel 29 receives a selection operation of selecting an image capturing protocol including, for example, the standing position mode related to the image capturing of the subject P in the standing position, a seated position mode related to the image capturing of the subject P in a seated position, or the supine position mode related to the image capturing of the subject P in a supine position. The operation panel 29 is provided, for example, on the support column 13.

The high-voltage generator 31 includes electric circuitry such as a transformer and a rectifier, and generates a high voltage to be applied to the X-ray tube 17 and a filament current to be supplied to the X-ray tube 17. In addition, the high-voltage generator 31 performs control of an output voltage corresponding to X-rays emitted by the X-ray tube 17. The high-voltage generator 31 may employ a transformer method, or may employ an inverter method.

The high-voltage generator 31 may be provided on the rotational frame 21, or may be provided on the main frame side of the gantry 11.

A wedge (not illustrated) is a filter for adjusting an X-ray intensity of X-rays emitted from the X-ray tube 17. Specifically, the wedge is a filter that passes X-rays emitted from the X-ray tube 17 and attenuates the X-rays such that the X-rays emitted from the X-ray tube 17 toward the subject P have a predefined distribution.

The wedge is, for example, a wedge filter or a bow-tie filter, and is a filter made by processing aluminum to have a predetermined target angle and a predetermined thickness.

A collimator (not illustrated) is a lead plate or the like for narrowing down X-rays having passed through the wedge into an X-ray irradiation range, and a slit is formed by a combination of a plurality of lead plates or the like.

The DAS 33 includes an amplifier that performs amplification processing on an electric signal output from each X-ray detection element of the X-ray detector 19, and an analog-to-digital (A/D) converter that converts an electric signal into a digital signal, to generate detection data. The detection data generated by the DAS 33 is transferred to the console device 100. Here, the detection data collected by emitting X-rays toward the subject P is an example of first detection data.

The console device 100 includes a memory 101, a display 103, an input interface 105, and the processing circuitry 107. Data communication between the memory 101, the display 103, the input interface 105, and the processing circuitry 107 is performed, for example, via a bus.

The memory 101 is a storage device such as a hard disk drive (HDD), a solid state drive (SSD), or an integrated circuit storage device that stores various types of information. The memory 101 stores, for example, parameter information 1011 to be described below. In addition, the memory 101 stores, for example, projection data and reconstructed image data.

Aside from the HDD, the SSD, or the like, the memory 101 may be a drive device for reading and writing various types of information from and into a portable storage medium such as a compact disc (CD), a digital versatile disc (DVD), or a flash memory, a semiconductor memory element such as a RAM, or the like.

A storage region of the memory 101 may be provided in the console device 100, or may be provided in an external storage device connected via a network. In addition, the memory 101 stores a control program according to the present embodiment. The memory 101 stores volume data and the like generated through pre-scanning and main scanning.

The display 103 displays various types of information. For example, the display 103 outputs a medical image (CT image) generated by the processing circuitry 107, a graphical user interface (GUI) for receiving various operations from the user, and the like.

As the display 103, for example, a liquid crystal display (LCD), a cathode ray tube (CRT) display, an organic electroluminescence (EL) display (OELD), a plasma display, or any other display can be appropriately used.

The display 103 may be provided in the gantry device 10. In addition, the display 103 may be a desktop-type display, or may be formed by a tablet terminal or the like that can wirelessly communicate with the main body of the console device 100. The display 103 corresponds to a display unit.

The input interface 105 receives various input operations from the user, converts the received input operations into electric signals, and outputs the electric signals to the processing circuitry 107. For example, the input interface 105 receives a collection condition to be used in collecting projection data, a reconstruction condition to be used in reconstructing a CT image, an image processing condition to be used in generating a postprocessed image from a CT image, and the like from the user.

As the input interface 105, for example, a mouse, a keyboard, a trackball, a switch, a button, a joystick, a touch pad, a touch panel display, and the like can be appropriately used.

In the present embodiment, the input interface 105 is not limited to an input interface including a physical operational component such as a mouse, a keyboard, a trackball, a switch, a button, a joystick, a touch pad, or a touch panel display.

For example, electric signal processing circuitry that receives an electric signal corresponding to an input operation from an external input device provided separately from the device, and outputs the received electric signal to processing circuitry 44 is also included in the examples of the input interface 105.

The input interface 105 may be provided in the gantry device 10. In addition, the input interface 105 may be formed by a tablet terminal or the like that can wirelessly communicate with the main body of the console device 100. The input interface 105 corresponds to an input unit.

The processing circuitry 107 controls operations of the entire X-ray CT apparatus 1 in accordance with an electric signal of an input operation output from the input interface 105. For example, the processing circuitry 107 includes, as hardware resources, a processor such as a CPU, an MPU, or a graphics processing unit (GPU), and a memory such as a ROM or a RAM.

By a processor that executes a program loaded in a memory, the processing circuitry 107 executes a system control function 111, a parameter calculation function 113, a determination function 115, a preprocessing function 117, a reconstruction function 119, and an image processing function 121.

Here, the system control function 111 is an example of a reception unit. The parameter calculation function 113 is an example of a calculation unit and a storage control unit. The determination function 115 is an example of an identification unit and a determination unit. The reconstruction function 119 is an example of a generation unit.

Each of the system control function 111, the parameter calculation function 113, the determination function 115, the preprocessing function 117, the reconstruction function 119, and the image processing function 121 is not necessarily implemented by a single piece of processing circuitry. Processing circuitry may be formed by combining a plurality of independent processors, and each of the system control function 111, the preprocessing function 117, the reconstruction function 119, and the image processing function 121 may be implemented by each processor executing a program.

The system control function 111 controls each function of the processing circuitry 107 based on an input operation received from the user via the input interface 105.

Specifically, the system control function 111 reads out a control program stored in the memory 101, loads the control program into a memory in the processing circuitry 107, and controls each component of the X-ray CT apparatus 1 in accordance with the loaded control program. For example, the system control function 111 controls each function of the processing circuitry 107 based on an input operation received from the user via the input interface 105.

In the present embodiment, the input operation includes selection input for an image capturing protocol. The image capturing protocol includes information indicating whether an image capturing mode is the standing position mode or the supine position mode. The image capturing mode is an example of posture information, and is also an example of gantry state information.

The parameter calculation function 113 calculates an image quality adjustment parameter to be used in reconstruction processing, for each image capturing mode. For example, the parameter calculation function 113 executes scanning of a phantom for each mode of the standing position mode and the supine position mode.

Based on detection data obtained by scanning of the phantom (hereinafter also referred to as raw data), the parameter calculation function 113 calculates correction data and an X-ray tube alignment (alignment correction value) included in the image quality adjustment parameter for each of the standing position mode and the supine position mode. Raw data in this case is an example of second detection data.

The correction data is data for correcting image quality of a reconstructed image, based on pixel values of water and air. For example, the correction data is calculated from a difference between a water CT value and an air CT value calculated from raw data obtained by scanning a phantom including water and air, and an ideal water CT value and an ideal air CT value. The correction data is calculated for each X-ray CT apparatus 1 for each of the water and air.

The alignment correction value is data for correcting an effect on a reconstructed image caused by a difference between a designed position of the X-ray tube 17 relative to the X-ray detector 19 (hereinafter also referred to as a design position), and an actually-arranged position of the X-ray tube 17 relative to the X-ray detector 19 (hereinafter also referred to also as an actual position).

For example, the alignment correction value is calculated based on a difference between raw data obtained by scanning of a phantom and raw data expected to be obtained in a case where the X-ray tube 17 is arranged at the design position. The alignment correction value is calculated for each X-ray CT apparatus 1 in a front-back direction and a left-right direction.

In addition, the parameter calculation function 113 sets (presets) an image quality parameter included in an image quality adjustment parameter for each of the standing position mode and the supine position mode.

The image quality parameter is a set of various parameters for determining the image quality of a reconstructed image. Examples of the image quality parameter include, for example, brightness, contrast, and the like, and may also include a method for adjusting brightness, contrast, or the like. The image quality parameter is preset for each X-ray CT apparatus 1. The image quality parameter may be preset for each image quality that can be designated by the user.

Here, because the influence of gravity on the X-ray tube 17 and the X-ray detector 19 inside the gantry 11 varies between a case where image capturing is executed in the supine position mode and a case where image capturing is executed in the standing position mode, a difference may arise in raw data.

For this reason, even when image capturing is performed by the same X-ray CT apparatus 1 under identical image capturing conditions except for the image capturing mode, and reconstruction processing is performed using the same image quality adjustment parameter, a difference in image quality, such as CT value deviation or noise generation, may arise depending on whether the image capturing mode is the standing position mode or the supine position mode. Thus, a reconstructed image with image quality desired by the user may not be obtained depending on the image capturing mode.

Thus, the X-ray CT apparatus 1 according to the present embodiment reduces a difference in the image quality that arises due to a difference in the image capturing mode by calculating and storing an image quality adjustment parameter for each image capturing mode, and using an image quality adjustment parameter suitable for the image capturing mode.

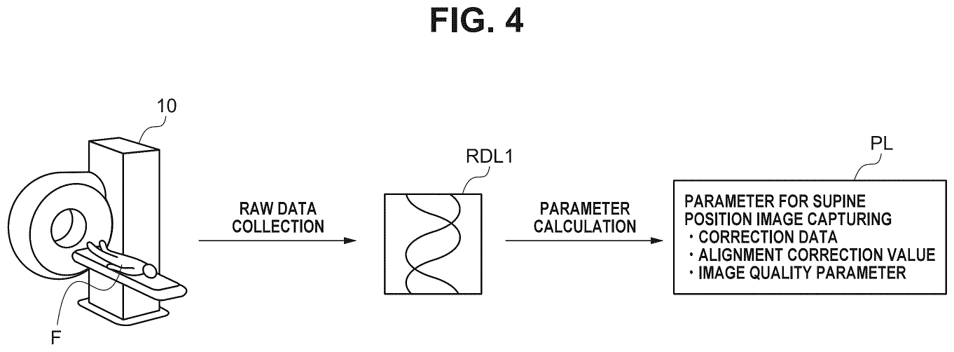

Here, FIG. 4 is a diagram illustrating an example of calculation processing of an image quality adjustment parameter for the supine position mode. As illustrated in FIG. 4, the system control function 111 controls each function of the processing circuitry 107, and collects raw data RDL1 by executing scanning of a phantom F in the supine position mode. In FIG. 4, the phantom F having a shape imitating a human is scanned in the supine position mode, but the shape of the phantom F scanned in the supine position mode is not limited to a human shape.

Subsequently, the parameter calculation function 113 calculates correction data and an alignment correction value as an image quality adjustment parameter PL for the supine position mode based on the raw data RDL1 collected by executing the scanning of the phantom F in the supine position mode. Furthermore, the parameter calculation function 113 presets an image quality parameter corresponding to the supine position mode as the image quality adjustment parameter PL for the supine position mode.

Further, the parameter calculation function 113 stores, in the parameter information 1011 in the memory 101, information indicating that the image capturing mode is the supine position mode in association with the calculated or preset image quality adjustment parameter PL.

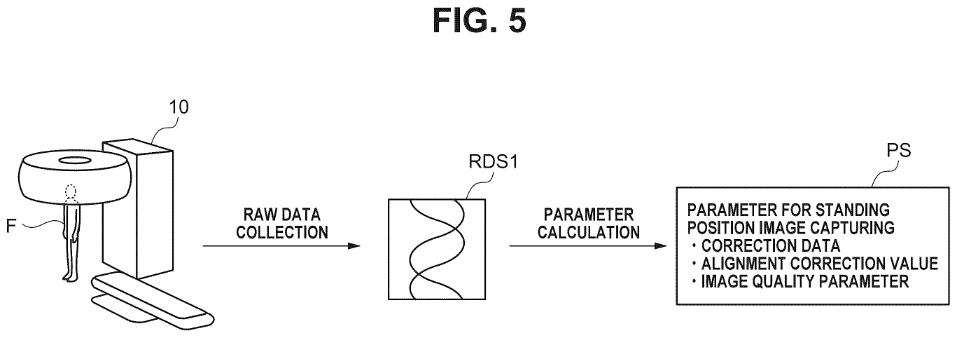

FIG. 5 is a diagram illustrating an example of calculation processing of an image quality adjustment parameter for the standing position mode. As illustrated in FIG. 5, the system control function 111 controls each function of the processing circuitry 107, and collects raw data RDS1 by executing scanning of the phantom F in the standing position mode. In FIG. 5, the phantom F having a shape imitating a human is scanned in the standing position mode, but the shape of the phantom F scanned in the standing position mode is not limited to a human shape.

Subsequently, the parameter calculation function 113 calculates correction data and an alignment correction value as an image quality adjustment parameter PS for the standing position mode based on the raw data RDS1 collected by executing the scanning of the phantom F in the standing position mode. Furthermore, the parameter calculation function 113 presets an image quality parameter corresponding to the standing position mode as the image quality adjustment parameter PS for the standing position mode.

Further, the parameter calculation function 113 stores, in the parameter information 1011 in the memory 101, information indicating that the image capturing mode is the standing position mode in association with the calculated or preset image quality adjustment parameter PS.

The description will now continue with reference to FIG. 1. The determination function 115 determines an image quality adjustment parameter to be used in the reconstruction processing based on the image capturing posture of the subject P.

For example, the determination function 115 identifies whether an image capturing mode included in the image capturing protocol, for which the selection input has been received by the system control function 111, is the standing position mode or the supine position mode. The determination function 115 refers to the parameter information 1011 in the memory 101, and determines an image quality adjustment parameter associated with the identified image capturing mode as an image quality adjustment parameter to be used in the reconstruction processing.

For example, the preprocessing function 117 generates data obtained by performing preprocessing such as logarithmic conversion processing, offset correction processing, inter-channel sensitivity correction processing, and beam hardening correction on raw data output from the DAS 33. The preprocessed data may also be referred to as projection data.

The reconstruction function 119 generates a reconstructed image by executing the reconstruction processing on the projection data.

For example, the reconstruction function 119 performs processing of correcting the effect on a reconstructed image caused by a difference between the design position and the actual position, on the projection data generated by the preprocessing function 117.

Specifically, the reconstruction function 119 generates corrected projection data obtained by correcting the effect on the reconstructed image by the difference between the design position and the actual position based on the projection data generated by the preprocessing function 117 and the alignment correction value included in the image quality adjustment parameter determined by the determination function 115. The above-described processing of correcting the effect on the reconstructed image by the difference between the design position and the actual position may be performed by the preprocessing function 117.

Furthermore, the reconstruction function 119 generates CT image data by performing the reconstruction processing that uses a filtered back projection (FBP) method, a successive approximation reconstruction method, or the like on the corrected projection data. In other words, the reconstruction function 119 generates a reconstructed image based on output from the image capturing system.

In addition, for example, the reconstruction function 119 performs processing of correcting a pixel value of each pixel on the reconstructed image.

Specifically, the reconstruction function 119 performs the processing of correcting a pixel value of each pixel on the reconstructed image, based on correction data and an image quality parameter included in the image quality adjustment parameter determined by the determination function 115. The above-described processing of correcting the pixel value of each pixel on the reconstructed image may be performed by the image processing function 121 to be described below.

The reconstruction function 119 stores data of a reconstructed image obtained by correcting the pixel value of each pixel on the reconstructed image, in the memory 101.

The image processing function 121 performs various types of image processing on a CT image reconstructed by the reconstruction function 119. For example, the image processing function 121 generates a display image by performing three-dimensional image processing such as volume rendering, surface volume rendering, image value projection processing, multi-planar reconstruction (MPR) processing, and curved MPR (CPR) processing on the CT image.

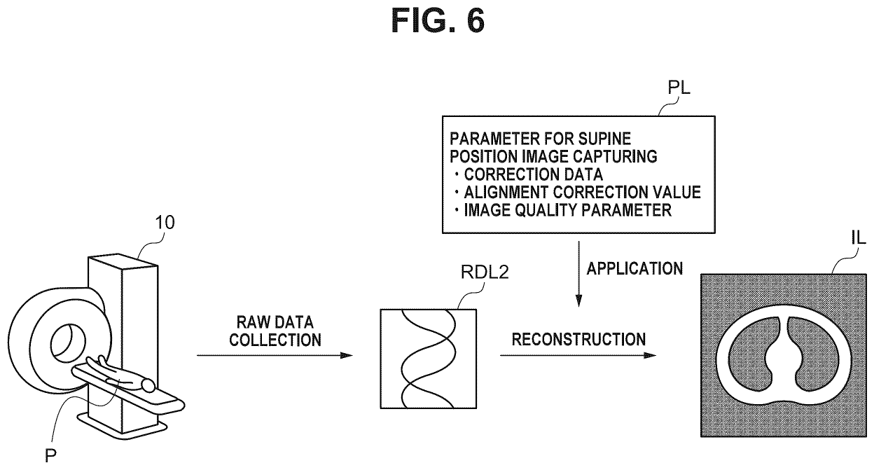

Here, FIG. 6 is a diagram illustrating an example of determination of an image quality adjustment parameter in the supine position mode, and reconstruction processing to which the determined image quality adjustment parameter is applied. First, the determination function 115 identifies that an image capturing mode is the supine position mode based on the image capturing protocol, for which the selection input has been received by the system control function 111.

Further, as illustrated in FIG. 6, the system control function 111 controls each function of the processing circuitry 107, and collects raw data RDL2 by executing the scanning of the subject P in the supine position mode. The preprocessing function 117 generates projection data of the subject P scanned in the supine position mode, based on the raw data RDL2.

The reconstruction function 119 generates a reconstructed image IL by performing reconstruction processing on the projection data using correction data, an alignment correction value, and an image quality parameter included in the image quality adjustment parameter determined by the determination function 115.

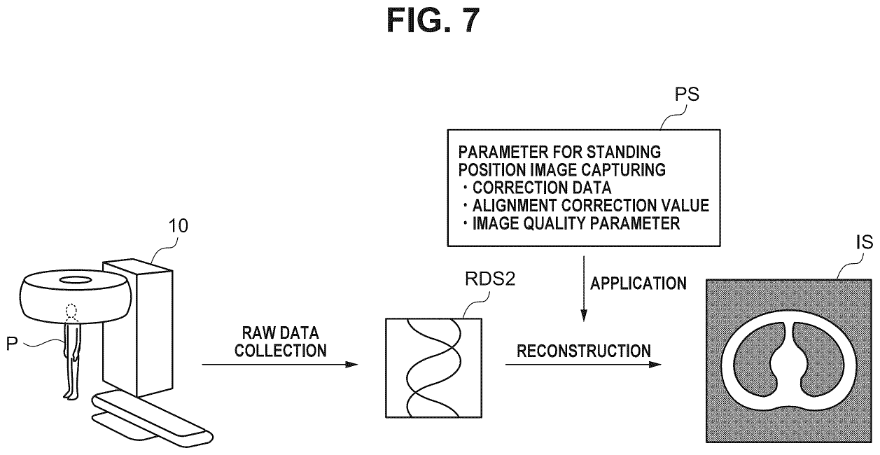

FIG. 7 is a diagram illustrating an example of determination of an image quality adjustment parameter for the standing position mode, and reconstruction processing to which the determined image quality adjustment parameter is applied. First, the determination function 115 identifies that an image capturing mode is the standing position mode, from the image capturing protocol of which the selection input has been received by the system control function 111.

Further, as illustrated in FIG. 7, the system control function 111 controls each function of the processing circuitry 107, and collects raw data RDS2 by executing the scanning of the subject P in the standing position mode. The preprocessing function 117 generates projection data of the subject P scanned in the standing position mode, based on the raw data RDS2.

The reconstruction function 119 generates a reconstructed image IS by performing reconstruction processing on the projection data using correction data, an alignment correction value, and an image quality parameter included in the image quality adjustment parameter determined by the determination function 115.

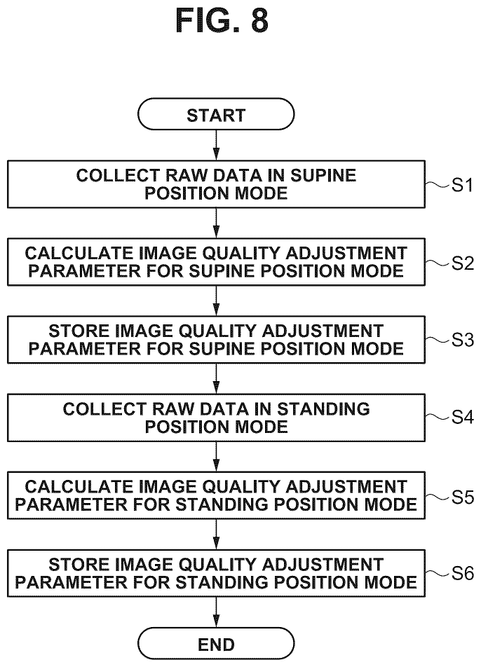

Hereinafter, processing to be executed by the X-ray CT apparatus 1 according to the present embodiment will be described. First, calculation and storing processing of the image quality adjustment parameter will be described. FIG. 8 is a flowchart illustrating an example of the calculation and storing processing of the image quality adjustment parameter executed by the X-ray CT apparatus 1 according to an embodiment. As an example, the calculation and storing processing of the image quality adjustment parameter is executed at the time of installation of the X-ray CT apparatus 1, at the time of maintenance work thereof, and the like.

First, in step S1, the system control function 111 collects raw data for calculating the image quality adjustment parameter in the supine position mode. For example, the system control function 111 controls each function of the processing circuitry 107, executes the scanning of the phantom F in the supine position mode, and collects the raw data for calculating the image quality adjustment parameter for the supine position mode.

Subsequently, in step S2, the parameter calculation function 113 calculates an image quality adjustment parameter for the supine position mode.

For example, the parameter calculation function 113 calculates correction data and an alignment correction value as the image quality adjustment parameter for the supine position mode, based on the raw data for calculating the image quality adjustment parameter for the supine position mode that has been collected in step S1. In addition, the parameter calculation function 113 presets an image quality parameter corresponding to the supine position mode as the image quality adjustment parameter for the supine position mode.

Subsequently, in step S3, the parameter calculation function 113 stores the image quality adjustment parameter for the supine position mode. For example, the parameter calculation function 113 stores, in the parameter information 1011 in the memory 101, information indicating that the image capturing mode is the supine position mode in association with the image quality adjustment parameter calculated in step S2.

Subsequently, in step S4, the system control function 111 collects raw data for calculating the image quality adjustment parameter in the standing position mode. For example, the system control function 111 controls each function of the processing circuitry 107, executes the scanning of the phantom F in the standing position mode, and collects the raw data for calculating the image quality adjustment parameter for the standing position mode.

Subsequently, in step S5, the parameter calculation function 113 calculates an image quality adjustment parameter for the standing position mode.

For example, the parameter calculation function 113 calculates correction data and an alignment correction value as the image quality adjustment parameter for the standing position mode, based on the raw data for calculating the image quality adjustment parameter for the standing position mode that has been collected in step S4. In addition, the parameter calculation function 113 presets an image quality parameter corresponding to the standing position mode as the image quality adjustment parameter for the standing position mode.

Subsequently, in step S6, the parameter calculation function 113 stores the image quality adjustment parameter for the standing position mode, and the processing ends. For example, the parameter calculation function 113 stores, in the parameter information 1011 in the memory 101, information indicating that the image capturing mode is the standing position mode in association with the image quality adjustment parameter calculated in step S5.

After the series of processes in steps S4 to S6 is executed, the series of processes in steps S1 to S3 may be executed. Alternatively, the series of processes in steps S1 to S3 and the series of processes in steps S4 to S6 may be executed separately on different occasions.

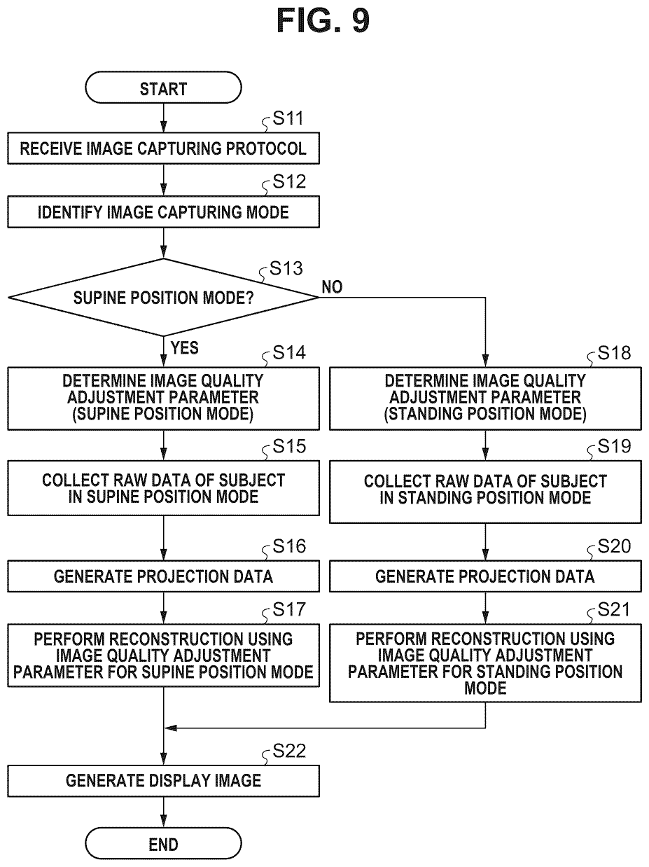

Next, determination and reconstruction processing of the image quality adjustment parameter will be described. FIG. 9 is a flowchart illustrating an example of the determination and reconstruction processing of the image quality adjustment parameter executed by the X-ray CT apparatus 1 according to an embodiment.

First, in step S11, the system control function 111 receives the selection input for an image capturing protocol from the user. The image capturing protocol includes information indicating whether an image capturing mode is the supine position mode or the standing position mode.

Subsequently, in step S12, the determination function 115 identifies the image capturing mode of the subject P. For example, the determination function 115 identifies the image capturing mode of the subject P from information indicating whether the image capturing mode is the supine position mode or the standing position mode that is included in the image capturing protocol received in step S11. Subsequently, in step S13, the determination function 115 determines whether the identified image capturing mode is the supine position mode or the standing position mode.

In a case where the image capturing mode is the supine position mode (YES in step S13), the processing proceeds to step S14. In step S14, the determination function 115 refers to the parameter information 1011 in the memory 101, and determines an image quality adjustment parameter associated with the supine position mode as the image quality adjustment parameter to be used in reconstruction processing.

Subsequently, in step S15, the system control function 111 collects raw data of the subject P in the supine position mode. For example, the system control function 111 controls each function of the processing circuitry 107, executes scanning of the subject P in the supine position mode, and collects the raw data.

Subsequently, in step S16, the preprocessing function 117 generates projection data. For example, the preprocessing function 117 generates the projection data by performing preprocessing such as logarithmic conversion processing, offset correction processing, inter-channel sensitivity correction processing, beam hardening correction, and the like on the raw data collected in step S15.

Subsequently, in step S17, the reconstruction function 119 performs reconstruction processing using the image quality adjustment parameter for the supine position mode. For example, the reconstruction function 119 generates a reconstructed image by executing reconstruction processing including various types of correction that use the image quality adjustment parameter for the supine position mode that has been determined in step S14, on the projection data generated in step S16. After step S17, the processing proceeds to step S22 to be described below.

On the other hand, in a case where the image capturing mode is the standing position mode (NO in step S13), the processing proceeds to step S18. In step S18, the determination function 115 refers to the parameter information 1011 in the memory 101, and determines an image quality adjustment parameter associated with the standing position mode as the image quality adjustment parameter to be used in the reconstruction processing.

Subsequently, in step S19, the system control function 111 collects raw data of the subject P in the standing position mode. For example, the system control function 111 controls each function of the processing circuitry 107, executes scanning of the subject P in the standing position mode, and collects the raw data.

Subsequently, in step S20, the preprocessing function 117 generates projection data. For example, the preprocessing function 117 generates the projection data by performing preprocessing such as logarithmic conversion processing, offset correction processing, inter-channel sensitivity correction processing, beam hardening correction, and the like on the raw data collected in step S19.

Subsequently, in step S21, the reconstruction function 119 performs reconstruction processing using the image quality adjustment parameter for the standing position mode. For example, the reconstruction function 119 generates a reconstructed image by executing reconstruction processing including various types of correction that use the image quality adjustment parameter for the standing position mode that has been determined in step S18, on the projection data generated in step S20.

Subsequently, in step S22, the image processing function 121 generates a display image, and the processing ends. For example, the image processing function 121 generates the display image by performing the above-described three-dimensional image processing on the reconstructed image generated in step S21.

As described above, the X-ray CT apparatus 1 according to the present embodiment identifies the orientation of the gantry 11 having the opening 15 into which the subject P is to be inserted, determines the image quality adjustment parameter to be used in the reconstruction processing based on the identified orientation of the gantry 11 and the parameter information 1011 storing the orientation of the gantry 11 and an image quality adjustment parameter in association, and executes the reconstruction processing on raw data using the determined image quality adjustment parameter to generate a reconstructed image.

Here, when the reconstruction processing is performed using the same image quality adjustment parameter irrespective of the orientation of the gantry 11, for example, because the effect of gravity received by a unit in the gantry 11 (the X-ray tube 17 or the X-ray detector 19) varies depending on the orientation of the gantry 11, a difference may arise also in the image quality of a reconstructed image generated through the reconstruction processing. In contrast to this, the X-ray CT apparatus 1 according to the present embodiment can perform the reconstruction processing using an image quality adjustment parameter suitable for the orientation (for example, the supine position mode or the standing position mode) of the gantry 11. The X-ray CT apparatus 1 according to the present embodiment can thereby reduce a difference in image quality of a CT image that is attributed to a difference in image capturing posture of the subject P.

The above-described embodiment may be modified and implemented as appropriate by changing part of the configuration or functions of the X-ray CT apparatus 1. Thus, hereinafter, modified examples of the above-described embodiment will be described as other embodiments. Hereinafter, a difference from the above-described embodiment will be mainly described, and detailed description of points common to the already-described content will be omitted. In addition, modified examples described below may be implemented individually or in combination as appropriate.

### FIRST MODIFIED EXAMPLE

In the above-described embodiment, the configuration is described in which the determination and reconstruction processing of the image quality adjustment parameter is executed on an assumption that an image capturing mode is the standing position mode in a case where the image capturing posture of the subject P is the seated position, similarly to the case where the image capturing posture of the subject P is the standing position. In the present modified example, a configuration is described in which an image quality adjustment parameter for a seated position image capturing state (also referred to as a seated position mode) is calculated and stored, and reconstruction processing is executed using the image quality adjustment parameter corresponding to the seated position mode in a case where an image capturing posture of the subject P is the seated position.

In the present modified example, the system control function 111 executes the scanning of the phantom F in the seated position mode in addition to the standing position mode and the supine position mode at the time of calculation of the image quality adjustment parameter. The parameter calculation function 113 calculates an image quality adjustment parameter for the seated position mode (correction data and alignment correction value) or presets the image quality adjustment parameter (image quality parameter), based on raw data obtained by executing the scanning of the phantom F in the seated position mode.

The parameter calculation function 113 stores, in the parameter information 1011 in the memory 101, information indicating that the image capturing mode is the seated position mode in association with the calculated image quality adjustment parameter for the seated position mode.

In the present modified example, the image capturing protocol includes information indicating whether the image capturing mode is the supine position mode, the standing position mode, or the seated position mode. In a case where the image capturing mode included in the image capturing protocol received by the system control function 111 is the seated position mode, the determination function 115 refers to the parameter information 1011 in the memory 101, and determines an image quality adjustment parameter associated with the seated position mode as the image quality adjustment parameter to be used in the reconstruction processing.

In the present modified example, in a case where an image capturing posture of the subject P is the seated position, an image quality adjustment parameter calculated based on raw data obtained by scanning the phantom F in the seated position mode, which is different from the image quality adjustment parameter corresponding to the standing position mode, can be used in the reconstruction processing. In other words, according to the present modified example, it is possible to further reduce a difference in image quality of a CT image that is attributed to a difference in image capturing posture of the subject P.

### SECOND MODIFIED EXAMPLE

In the above-described embodiment, the configuration is described in which the image capturing posture includes the supine position (in which an installation surface of the support column 13 and a body axis of the subject P are parallel to each other) and the standing position or the seated position (in which the installation surface of the support column 13 and the body axis of the subject P are perpendicular to each other). In the present modified example, a configuration in which an image of the subject P can be captured by setting an angle formed by the installation surface of the support column 13 and the body axis of the subject P to any angle from 90° (the installation surface of the support column 13 and the body axis of the subject P are perpendicular to each other) to 180° (the installation surface of the support column 13 and the body axis of the subject P are parallel to each other) will be described.

In the present modified example, the couchtop movement mechanism 37 includes a mechanism that can change an angle of a placement surface of the subject P of the couchtop 30 relative to the installation surface of the support column 13 to any angle from 90° to 180°. The system control function 111 according to the present modified example receives, from the user, an input operation of a desired angle (hereinafter also referred to as a couchtop angle) of the couchtop 30 relative to the installation surface of the support column 13. As an example, the system control function 111 receives an input operation of inputting any angle from 90° to 180° in 5° increments.

The movement control circuitry 27 according to the present modified example performs control of changing an angle of the couchtop 30 with respect to the installation surface of the support column 13 (hereinafter also referred to as a couchtop angle) to any angle from 90° to 180° desired by the user.

In addition, when the movement control circuitry 27 changes the angle of the couchtop 30 to any angle from 90° to 180° desired by the user, the movement control circuitry 27 rotates the gantry 11 such that the opening 15 is perpendicular to the placement surface of the subject P of the couchtop 30.

In the present modified example, the system control function 111 executes the scanning of the phantom F for each couchtop angle (every 5° from 90° to 180° in the present modified example) at the time of calculation of an image quality adjustment parameter. The parameter calculation function 113 calculates an image quality adjustment parameter (correction data and alignment correction value) for each couchtop angle or presets the image quality adjustment parameter (image quality parameter), based on raw data obtained by executing the scanning of the phantom F for each couchtop angle.

The parameter calculation function 113 stores, in the parameter information 1011 in the memory 101, information indicating a couchtop angle and a calculated image quality adjustment parameter for the seated position mode in association with each other.

In the present modified example, an image capturing protocol includes the information indicating a couchtop angle. The determination function 115 refers to the parameter information 1011 in the memory 101, and determines an image quality adjustment parameter associated with the couchtop angle included in the image capturing protocol received by the system control function 111, as an image quality adjustment parameter to be used in reconstruction processing.

The parameter calculation function 113 may calculate an image quality adjustment parameter for each couchtop angle based on raw data collected at a plurality of predefined couchtop angles. For example, the system control function 111 may execute the scanning of the phantom F for each of the cases where the couchtop angles are 90°, 135°, and 180°.

In this case, the parameter calculation function 113 may predict an image quality adjustment parameter for each of the cases where the couchtop angles are 90° to 135°, and 135° to 180°, based on a calculation result of the image quality adjustment parameter for each of the cases where the couchtop angles are 90°, 135°, and 180°.

In this manner, by calculating the image quality adjustment parameter for each couchtop angle based on the raw data collected at the plurality of predefined couchtop angles, it is unnecessary to collect raw data at all angles, and it is possible to reduce processing load of calculation and storing processing of the image quality adjustment parameter.

In the present modified example, an image quality adjustment parameter suitable for a couchtop angle at which an image of the subject P is captured can be used in reconstruction processing. In other words, according to the present modified example, it is possible to reduce a difference in image quality of a CT image that is attributed to a difference in an image capturing posture of the subject P.

### THIRD MODIFIED EXAMPLE

In the above-described embodiment, a configuration is described in which the parameter calculation function 113 calculates the correction data for water and air for each image capturing mode. However, the parameter calculation function 113 may calculate correction data for either water or air for each image capturing mode.

For example, the parameter calculation function 113 may calculate correction data for water for each image capturing mode, and may calculate correction data for air only for the supine position mode (or standing position mode). In this case, the reconstruction function 119 may perform reconstruction using common correction data for air.

Further, for example, the parameter calculation function 113 may calculate correction data for air for each image capturing mode, and may calculate correction data for water only for the supine position mode (or standing position mode). In this case, the reconstruction function 119 may perform reconstruction using common correction data for water.

According to the present modified example, it is possible to reduce a processing load related to calculation processing of an image quality parameter.

### FOURTH MODIFIED EXAMPLE

In the above-described embodiment, the configuration is described in which the parameter calculation function 113 calculates the image quality adjustment parameter for each of the supine position mode and the standing position mode based on the raw data obtained by executing the scanning of the phantom F in both the supine position mode and the standing position mode. In the present modified example, a configuration in which an image quality adjustment parameter is calculated for each of the supine position mode and the standing position mode based on raw data obtained by executing the scanning of the phantom F in either the supine position mode or the standing position mode will be described.

In the present modified example, when an image quality adjustment parameter is calculated, the system control function 111 collects raw data by executing the scanning of the phantom F only in the supine position mode. The parameter calculation function 113 calculates an image quality adjustment parameter for the supine position mode based on the collected raw data.

Here, in a case where the image capturing mode changes from the supine position mode to the standing position mode, it is possible to predict, by calculation, how the state of a unit in the gantry 11 (the X-ray tube 17, the X-ray detector 19, or the like) changes, i.e., how the position of the unit changes by the influence of gravity.

Based on the predicted change in the state of the unit in the gantry 11, the parameter calculation function 113 predicts changes in water and air CT values and a position change of the X-ray tube 17 relative to the X-ray detector 19 that occur in a case where the image capturing mode changes from the supine position mode to the standing position mode. The parameter calculation function 113 calculates correction data and an alignment correction value for the standing position mode based on prediction results of the changes in water and air CT values and the position change of the X-ray tube 17 relative to the X-ray detector 19.

Alternatively, the system control function 111 may collect the raw data by executing the scanning of the phantom F only in the standing position mode.

According to the present modified example, it is possible to reduce processing load of processing of collecting raw data in calculating an image quality adjustment parameter.

### FIFTH MODIFIED EXAMPLE

In the above-described embodiment, the configuration is described in which the type of object that is other than the subject P and that exists in the opening 15 of the gantry 11 at the time of image capturing is the couchtop 30 or the patient fixation pole 30a. In the present modified example, a configuration in which an image quality adjustment parameter is switched in accordance with a substance other than the subject P that is to be inserted into the opening 15 of the gantry 11 and that is other than the couchtop 30 or the patient fixation pole 30a will be described.

In the present modified example, in the image capturing of the subject P in the supine position mode, an Angio couch may sometimes be used aside from the couchtop 30 (CT couch). The above-described couch is an example of a couch type to be used in the image capturing of the subject P in the supine position mode, and a couch (couchtop) of another type may be used in the image capturing of the subject P.

Here, for example, in a system capable of performing Angio-CT image capturing (Angio-CT mode) and normal CT image capturing (normal-CT mode), an object present in an X-ray tube path (between the X-ray tube 17 and the X-ray detector 19) differs between the Angio couch and the couchtop 30, between a case where the mode is the Angio-CT mode and a case where the mode is the normal-CT mode.

When the object present in the X-ray tube path differs, even when image capturing of the subject P is performed using the same system under the same image capturing condition, image quality of the obtained CT image may vary.

Thus, in the present modified example, the system control function 111 executes the scanning of the phantom F for each image capturing mode and for each type of object present in the X-ray tube path at the time of calculating an image quality adjustment parameter.

The parameter calculation function 113 calculates an image quality adjustment parameter for each image capturing mode and for each type of object present in the X-ray tube path (correction data and alignment correction value) or presets the image quality adjustment parameter (image quality parameter), based on raw data obtained by executing the scanning of the phantom F for each image capturing mode and for each type of object present in the X-ray tube path.

The parameter calculation function 113 stores, in the parameter information 1011 in the memory 101, information indicating an image capturing mode and information indicating the type of object present in the X-ray tube path in association with the calculated image quality adjustment parameter.

In the present modified example, an image capturing protocol includes information indicating the type of object present in the X-ray tube path. The information indicating the type of object present in the X-ray tube path is an example of object information and gantry state information. The determination function 115 refers to the parameter information 1011 in the memory 101, and determines an image quality adjustment parameter associated with a combination of an image capturing mode and the type of object present in the X-ray tube path that is included in the image capturing protocol received by the system control function 111, as an image quality adjustment parameter to be used in reconstruction processing.

A plurality of objects may be present in the X-ray tube path. In addition, the object present in the X-ray tube path may include clothes worn by the subject P, an infusion tube, or the like.

According to the present modified example, it is possible to reduce a difference in image quality of a CT image that is attributed to a difference in the type of object present in the X-ray tube path.

### SIXTH MODIFIED EXAMPLE

In the above-described embodiment, the configuration is described in which the reconstruction processing is performed by the console device 100. In the present modified example, an example of executing reconstruction processing by an external image processing apparatus different from the console device 100 will be described.

In the present modified example, processing circuitry of the external image processing apparatus includes a parameter calculation function 113, a determination function 115, a preprocessing function 117, a reconstruction function 119, and an image processing function 121.

The system control function 111 according to the present modified example transmits, to an image processing apparatus connected with the X-ray CT apparatus 1 via a network, information indicating a scan target (for example, information indicating whether the scan target is the phantom F or the subject P) and information indicating whether an image capturing mode is the supine position mode or the standing position mode in association with the collected raw data.

The parameter calculation function 113 of the image processing apparatus identifies an image capturing mode based on the information indicating whether the image capturing mode is the supine position mode or the standing position mode that is associated with the raw data that has been obtained by scanning the phantom F and transmitted from the X-ray CT apparatus 1.

The parameter calculation function 113 calculates an image quality adjustment parameter for the supine position mode and an image quality adjustment parameter for the standing position mode, and stores the image quality adjustment parameters in the parameter information 1011 in a memory of the image processing apparatus in association with the information indicating whether the image capturing mode is the supine position mode or the standing position mode.

The determination function 115 identifies the image capturing mode based on the information indicating whether the image capturing mode is the supine position mode or the standing position mode that is associated with the raw data that has been obtained by scanning the subject P and transmitted from the X-ray CT apparatus 1. The determination function 115 refers to the parameter information 1011 in the memory, and determines an image quality adjustment parameter corresponding to the identified image capturing mode as the image quality adjustment parameter to be used in reconstruction processing.

Because the processing executed by the preprocessing function 117, the reconstruction function 119, and the image processing function 121 is similar to that in the above-described embodiment, the description thereof will be omitted.

According to the present modified example, it is possible to reduce processing load of the console device 100.

According to at least one of the embodiment, modified examples, and the like described above, it is possible to reduce a difference in image quality of a CT image that is attributed to a difference in an image capturing posture of the subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography (CT) apparatus comprising processing circuitry configured to:
identify gantry state information indicating a state of a gantry, the gantry state information including at least one of orientation information indicating an orientation of the gantry having an opening, and object information indicating an object other than a subject, the object existing in the opening;
determine an image quality adjustment parameter to be used in reconstruction processing, based on the identified gantry state information, and parameter information associating the gantry state information with the image quality adjustment parameter including a plurality of parameters for determining image quality of a reconstructed image generated through reconstruction processing; and
generate the reconstructed image by executing the reconstruction processing on first detection data collected by emitting an X-ray toward the subject using the determined image quality adjustment parameter.

2. The X-ray CT apparatus according to claim 1, wherein the image quality adjustment parameter includes at least one of correction data for adjusting image quality of the reconstructed image based on pixel values of water and air, an alignment correction value indicating a shift between a designed position of an X-ray tube provided in the gantry and a position where the X-ray tube is actually arranged, and an image quality parameter being a parameter set for determining the image quality of the reconstructed image.

3. The X-ray CT apparatus according to claim 2,
wherein the gantry state information includes the orientation information, and
wherein the processing circuitry is further configured to:
calculate at least one of the correction data and the alignment correction value based on second detection data collected by emitting an X-ray toward a phantom, for each orientation of the gantry; and
store, in the parameter information, at least one of the calculated correction data and the alignment correction value as the image quality adjustment parameter in association with the orientation information indicating the orientation of the gantry for which the second detection data is collected.

4. The X-ray CT apparatus according to claim 3, wherein the processing circuitry stores, in the parameter information, the image quality parameter as the image quality parameter in association with the orientation information indicating the orientation of the gantry, for each orientation of the gantry.

5. The X-ray CT apparatus according to claim 3, wherein the orientation information includes a supine position mode indicating an orientation of the gantry in a case where an image of the subject in a supine position state is captured, and a standing position mode indicating the orientation of the gantry in a case where an image of the subject in a standing position state is captured.

6. The X-ray CT apparatus according to claim 5, wherein the processing circuitry performs processing of calculating the image quality parameter associated with orientation information for other modes including the standing position mode based on the image quality parameter associated with the supine position mode, or processing of calculating the image quality parameter associated with orientation information for other modes including the supine position mode based on the image quality parameter associated with the standing position mode.

7. The X-ray CT apparatus according to claim 1, wherein the processing circuitry is further configured to:
receive an input of an image capturing protocol including at least an image capturing condition under which an image of the subject is captured and the gantry state information; and
identify the gantry state information based on the received image capturing protocol.

8. The X-ray CT apparatus according to claim 1,
wherein the gantry state information includes the object information, and
wherein the object includes a couchtop on which the subject in a supine position state is placed during image capturing, and a columnar subject holding tool that supports the subject in a standing position state during image capturing.

9. The X-ray CT apparatus according to claim 8,
wherein the X-ray CT apparatus is capable of performing Angio-CT image capturing of the subject, and
wherein the object further includes an Angio couch on which the subject is placed during Angio-CT image capturing of the subject.

10. An image processing method for performing reconstruction processing based on detection data collected by an X-ray computed tomography (CT) apparatus, the image processing method comprising:
identifying gantry state information indicating a state of a gantry, the gantry state information including at least one of orientation information indicating an orientation of the gantry having an opening into which a subject is to be inserted, and object information indicating an object other than the subject, the object existing in the opening;
determining an image quality adjustment parameter to be used in reconstruction processing, based on the identified gantry state information, and parameter information associating the gantry state information with the image quality adjustment parameter including a plurality of parameters for determining image quality of a reconstructed image generated through reconstruction processing; and
generating the reconstructed image by executing the reconstruction processing on first detection data collected by emitting an X-ray toward the subject using the determined image quality adjustment parameter.
